(19) United States
(12) Patent Application Publication
(10) Pub. No.: US 2026/0007511 A1
(43) Pub. Date: Jan. 8, 2026

(54) SYSTEMS, METHODS, AND DEVICES FOR SECURING SUTURES IN THE EYE

(71) Applicant: David Youngil Rhee, Long Island City, NY (US)

(72) Inventor: David Youngil Rhee, Long Island City, NY (US)

(21) Appl. No.: 19/240,674

(22) Filed: Jun. 17, 2025

Related U.S. Application Data
(60) Provisional application No. 63/668,616, filed on Jul. 8, 2024.

(51) Int. Cl. A61F 2/16; A61B 17/04

(52) U.S. Cl. CPC A61F 2/1662; A61B 17/0401; A61B 17/0469; A61B 2017/0409; A61B 2017/047

(57) ABSTRACT

The embodiments herein are directed to systems for securing a suture in an eye, the systems comprising an anchor. The anchor may be inserted into an incision in the eye with a trocar. The anchor may include a conduit. A suture may be inserted through the conduit from inside the eye and the suture may be secure to the anchor. The system may comprise a plug. The plug may be inserted into the anchor to secure the suture to the anchor. The plug may comprise a cutout. The suture may be inserted through the cutout.

### INCORPORATION BY REFERENCE TO ANY PRIORITY APPLICATIONS

This application claims priority to U.S. Provisional Patent Application No. 63/668,616, filed Jul. 8, 2024, the entirety of which is incorporated herein by reference. Any and all applications for which a foreign or domestic priority claim is identified in the Application Data Sheet as filed with the present application are hereby incorporated by reference under 37 CFR 1.57.

### BACKGROUND

### Field

The present disclosure relates to systems, methods, and device, for securing sutures in the eye, such as during an intraocular lens surgical operation.

### Description

Typically, intraocular lenses (IOLs) are secured in place during surgical procedures by securing sutures to the eye (e.g., the cornea and/or the sclera). In order to secure the sutures to the eye, ends of the sutures are inserted through incisions in the eye so the ends of the sutures extend out of the eye and the ends of the sutures are tied together. Typically, each end of the sutures extend through separate incisions. Therefore, the sutures may extend across a portion of the eye between the incisions. One drawback of the traditional method of securing sutures is that the sutures can erode nearby tissues, break, and can cause inflammation, discomfort, and visual disturbances. Additionally, sutured IOLs are sometimes prone to long-term stability issues like decentration or dislocation due to factors such as suture loosening, leading to suboptimal visual outcomes and potentially requiring additional surgical interventions for repositioning or replacement of the IOL. Furthermore, securing IOLs in place with sutures can require meticulous surgical skill and precision, particularly in eyes with compromised anatomy or limited surgical access, making the procedure technically challenging. If not precisely aligned or tensioned placement of sutures in the cornea or sclera can induce astigmatism, adversely affecting postoperative visual acuity and quality of vision. Other techniques, such as the Yamane technique, attempt to addresses these drawbacks by securing IOLs in place without sutures. However, these techniques often require more surgical skill and precision in order to properly secure the IOLs in place.

### SUMMARY

For purposes of this summary, certain aspects, advantages, and novel features of the invention are described herein. It is to be understood that not all such advantages necessarily may be achieved in accordance with any particular embodiment of the invention. Thus, for example, those skilled in the art will recognize that the invention may be embodied or carried out in a manner that achieves one advantage or group of advantages as taught herein without necessarily achieving other advantages as may be taught or suggested herein.

In accordance with one aspect of the disclosure, a method for securing a suture to an eye can include the steps of: inserting a trocar through the eye to form an incision in the eye, a cannula can be positioned over the trocar and an anchor can be positioned over the cannula such that the cannula is positioned in a conduit extending through the anchor; removing the trocar from the eye such that the anchor and the cannula remain in the incision; removing the cannula from the conduit in the anchor; inserting an end of the suture through the anchor; and securing the suture to the anchor.

In some aspects, securing the suture to the anchor can further include inserting the suture through a cutout in a plug of the anchor; and inserting the plug into the conduit in the anchor;

In some aspects, securing the suture to the anchor can further include knotting the end of the suture and/or trimming the suture.

In some aspects, the cutout can be tapered along a depth of the cutout.

In some aspects, the cutout can configured to clamp onto the suture when the plug is inserted into the anchor.

In some aspects, the method can further include inserting a second trocar through the eye to form a second incision in the eye, a second cannula can be positioned over the second trocar and a second anchor can positioned over the second cannula such that the second cannula is positioned in a conduit extending through the second anchor; removing the second trocar from the eye such that the second anchor and the second cannula remain in the second incision; removing the second cannula from the conduit in the second anchor; inserting a second end of the suture through the second anchor; and securing the suture to the anchor.

In accordance with one aspect of the disclosure, an anchor for securing a suture to an eye can include a sleeve including a conduit extending through the sleeve; a collar extending from a proximal end of the sleeve, the sleeve can be configured to receive a plug in the conduit extending through the sleeve, the plug can be configured to couple the suture to the anchor when the sleeve is positioned in an incision in the eye.

In some aspects, the sleeve and/or the collar can include silicone.

In some aspects, the anchor can further include a projection extending into the conduit from an inner wall of the sleeve.

In some aspects, the projection can be configured to secure the anchor in the conduit of the sleeve.

In some aspects, the sleeve can be inserted into the incision in the eye with a trocar.

In some aspects, the trocar can include a 23-gauge trocar, a 25-gauge trocar, or a 27-gauge trocar.

In accordance with one aspect of the disclosure, a system for securing a suture to an eye can include an anchor configured to be inserted into an incision in the eye, the anchor can include a conduit; a plug configured to be inserted into the conduit, the plug can be configured to secure the suture in place when the plug is inserted into the conduit.

In some aspects, the anchor and the plug can include silicone.

In some aspects, the anchor can include a sleeve and a collar extending from the sleeve, and the conduit can extend through the sleeve.

In some aspects, the anchor can include a projection extending into the conduit, and the projection can be configured to secure the plug in the anchor.

In some aspects, the plug can include a recess configured to receive the projection when the anchor is positioned in the conduit.

In some aspects, a distal end of the plug can be inserted into the conduit first, and the plug can be tapered at the distal end.

In some aspects, the plug can include a cutout configured to receive the suture.

In some aspects, the cutout can extend through the plug from a proximal end of the plug to a distal end of the plug.

In some aspects, the plug can include a collar.

In some aspects, the collar can extend radially outward from a proximal end of the plug.

In some aspects, the collar can include a cutout.

Although several configurations, examples, and illustrations are disclosed below, it will be understood by those of ordinary skill in the art that the disclosure extends beyond the specifically disclosed configurations, examples, and illustrations and includes other uses of the disclosure. Configurations are described with reference to the accompanying figures, wherein like numerals refer to like elements throughout. The terminology used in the description presented herein is not intended to be interpreted in any limited or restrictive manner simply because it is being used in conjunction with a detailed description of some specific configurations of the disclosure. In addition, configurations can comprise several novel and inventive features. No single feature is solely responsible for its desirable attributes or is essential to practicing the disclosure herein described.

### BRIEF DESCRIPTION OF THE DRAWINGS

Throughout the drawings, reference numbers may be reused to indicate general correspondence between referenced elements. The drawings are provided to illustrate example embodiments and are not intended to limit the scope of the disclosure. A better understanding of the systems and methods described herein will be appreciated upon reference to the following description in conjunction with the accompanying drawings, wherein:

FIG. 1 illustrates a system for securing a suture in an eye according to some embodiments herein.

FIG. 2A illustrates an anchor of the system of FIG. 1A.

FIG. 2B illustrates a cross-section of the anchor of FIG. 2A.

FIG. 2C illustrates another embodiment of an anchor according to some embodiments herein.

FIG. 2D illustrates a cross-section of the anchor of FIG. 2C.

FIG. 3A illustrates a plug of the system of FIG. 1A.

FIG. 3B illustrates a top view of the plug of FIG. 3A.

FIG. 3C illustrates another embodiment of a plug according to some embodiments herein.

FIG. 3D illustrates another embodiment of a plug according to some embodiments herein.

FIG. 3E illustrates a top view of the plug of FIG. 3D.

FIG. 4A illustrates another embodiment of a plug according to some embodiments herein.

FIG. 4B illustrates the plug of FIG. 4A inserted into the anchor of FIG. 2A.

FIG. 4C illustrates another embodiment of a plug according to some embodiments herein.

FIG. 5 illustrates a trocar for inserting an anchor into an eye, with a cannula and the anchor positioned over the trocar according to some embodiments herein.

FIG. 6 illustrates an example method for performing an intraocular surgical operation with the system of FIG. 1 according to some embodiments herein.

### DETAILED DESCRIPTION

Although certain preferred embodiments and examples are disclosed below, inventive subject matter extends beyond the specifically disclosed embodiments to other alternative embodiments and/or uses and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present technology.

Cataract surgery is a common procedure performed to treat cataracts, which cloud the natural lens of the eye. Cataracts typically occur with age, but can also result from injury, certain medications, or medical conditions. During cataract surgery or subsequent lens replacement surgery, the cloudy lens or artificial lens is removed and replaced with an artificial lens called an intraocular lens (IOL).

Intraocular lenses (IOLs) can be secured in the eye through various techniques, depending on factors such as the condition of the eye's natural structures (e.g., lens capsule), the presence of any abnormalities or complications, and the surgeon's preference and expertise. In cases where the natural lens capsule is intact and sufficiently strong, the IOL can be placed inside the capsular bag, where the natural lens was located. The IOL is inserted through a small incision and positioned within the bag, where it remains securely in place due to the support provided by the remaining capsule.

When the natural lens capsule is compromised or absent, making capsular bag fixation challenging or impossible, scleral fixation may be utilized. Scleral fixation involves securing the IOL to the sclera, the white outer layer of the eye. The existing methods for scleral fixation include surgical techniques such as suturing and the Yamane technique. For example, a surgeon may suture the IOL in place or bury ends of the IOL into the sclera to secure the IOL in place.

During surgery, a trocar may be inserted into the eye to form incisions in the eye. A cannula may be positioned over the trocar and the cannula may remain in each incision after the trocar is removed. Trocars are sharp, pointed instruments designed to create precise entry points into the eye through the sclera. They are often used to create the initial scleral incisions for the placement of fixation sutures during IOL replacement surgery. Trocars come in various sizes and designs, and the choice of trocar depends on factors such as the surgical technique, the size of the incision required, and the surgeon's preference. During surgery, trocars are carefully inserted into the sclera at predetermined locations, typically guided by surgical markers or measurements, to ensure accurate placement of the fixation sutures and minimize the risk of complications. Surgical instruments may be inserted through the trocar in order to perform one or more steps of a surgical operation.

Cannulas may be inserted into the incisions made by the trocars. Cannulas are thin, hollow tubes used to maintain a clear passage for the introduction of instruments or fluids into the eye during surgery. For example, ends of the sutures may be inserted through the cannula such that the ends of the sutures extend out of the eye for manipulation by the surgeon. The ends of the sutures may be tied together to form knots, and the knots may be buried in the conjunctiva and/or the sclera. Burying the ends of the IOL into the sclera may be more difficult technically than suturing the IOL since, for example, the IOL may be more easily centered using sutures. However, the sutures may be coarse so the sutures may erode through the conjunctiva or irritate the eye, and the surgeon may eventually have to replace the sutures and attempt to re-approximate or re-close the conjunctiva. Additionally, if the conjunctiva is not opened during surgery, burying a knot in the sutures can be difficult. A surgeon may spend a several minutes trying to find a hole in the eye in which to bury the knot.

Suturing techniques utilize IOL haptics (supporting arms) to attach the IOL to the sclera using sutures, which are passed through small incisions in the sclera. The sutures provide stability and support for the IOL. However, Traditional sutured IOL techniques involved passing sutures through the sclera to secure the IOL in place. However, sutures are vulnerable to erosion of the surrounding tissue, breakage, or knot exposure over time. Suture-related complications could cause inflammation, discomfort, and even visual disturbances to patients. Furthermore, sutured IOLs are prone to decentration or dislocation over time. Dislocation could occur due to suture loosening, scleral thinning, or other factors, leading to suboptimal visual outcomes and potential complications that necessitate additional surgical interventions to reposition or replace the IOL. Traditional sutured IOL techniques often require meticulous surgical skill and precision to ensure proper placement and tensioning of the sutures.

The Yamane Technique is a relatively newer method that involves fixating the IOL haptics to the sclera without the use of sutures passing through the cornea. Instead, the haptics are threaded through scleral tunnels created using specialized needles, reducing the risk of corneal and conjunctival complications. Nevertheless, the Yamane technique requires a high level of surgical skill and experience, particularly in handling and manipulating intraocular tissues. Surgeons need specialized training and proficiency in the technique to achieve optimal outcomes and minimize complications. In addition, making adjustments to an IOL that has been secured using the Yamane technique may be extremely difficult or impossible. The systems, methods, and devices described herein may provide improvements in the existing methods of securing sutures within the eye.

In accordance with several embodiments, the systems, method, and devices described herein advantageously reduce or minimize an amount of exposed sutures, and facilitate easier insertion, securement, and adjustment of the sutures to the eye during insertion or replacement of IOLs. In some embodiments, an anchor and plug system for securing sutures in the eye during surgery may be used to provide stable fixation of sutures to intraocular tissues such as the sclera. In some embodiments, the anchor may comprise a small, biocompatible device designed to be inserted into the eye and securely engage with the sclera. In some embodiments, the anchor may comprise a textured or barbed surface to enhance its grip and prevent slippage once inserted. In some embodiments, the anchors may comprise various shapes and sizes, depending on the specific application and surgeon preference. In some embodiments, the anchor may substantially comprise a disk, cylinder, cone, or anchor with multiple arms or prongs. In some embodiments, the anchor may comprise of materials such as silicone, polypropylene, or other inert materials that are well-tolerated by intraocular tissues and do not induce inflammation or tissue reactions.

In some embodiments, during surgery, the anchor is carefully inserted into the desired location within the eye, such as the sclera or iris, using specialized insertion instruments such as a trocar and/or cannula. Once positioned, the anchor provides a stable point of attachment for the suture.

In some embodiments, the system may comprise a complementary device (e.g., plug) designed to securely hold the suture in place and prevent it from loosening or migrating from the anchor. In some embodiments, the plug may be comprised of a soft, flexible material that conforms to the shape of the anchor and provides a snug fit around the suture. In some embodiments, the plug may have features such as flanges or ridges to enhance its stability and prevent slippage once inserted into the anchor. In some embodiments, after the anchor is positioned within the eye and the suture is passed through the desired tissue, the plug may be inserted into the anchor to secure the suture in place. In some embodiments, the plug is advanced along the suture until it reaches the anchor, where it expands or compresses to grip the suture tightly. In some embodiments, the plug may be present in the anchor at the time that the anchor is inserted into the eye. In some embodiments, once the plug is in place, it effectively locks the suture in position, providing durable fixation and maintaining tension on the tissue as needed for the surgical procedure. In some embodiments, the anchor and plug system offers enhanced stability, reduced risk of suture loosening or migration, and improved control over tissue tension.

In some embodiments, the system may include an anchor. In some embodiments, the anchor (e.g., a sleeve) may be positioned over the cannula when the cannula is positioned over the trocar. In some embodiments, the anchor may be sized and/or shaped such that the anchor is compatible with any trocar and/or cannular commonly used for intraocular surgical procedures. For example, the anchor may be sized and/or shaped so the anchor may be positioned over a 23-gauge trocar and/or cannula, a 25-gauge trocar and/or cannula, a 27-gauge trocar and/or cannula, and/or any other size of trocar or cannula using for intraocular surgery. In some embodiments, the anchor may comprise silicone, polypropylene (PP), polyvinyl alcohol (PVA), ethylene vinyl acetate (EVA), and/or any other biocompatible material. In some embodiments, the trocar may be inserted through the eye without opening the conjunctiva before the trocar is inserted into the eye. In some embodiments, the trocar may be inserted into the eye with the cannula positioned over the trocar, and the anchor positioned over the cannula. In some embodiments, when the trocar is removed, the anchor and the cannula may remain in an incision formed by the trocar. In some embodiments, when the cannula is removed from the incision, the anchor may remain in the incision.

In some embodiments, the suture may be inserted through the anchor such that ends of the suture extend out of the eye from the anchor. In some embodiments, the ends of the suture may be coupled to the anchor in order to secure the suture to the eye and/or secure an IOL in place. In some embodiments, the suture may be coupled to a plug (e.g., insert). In some embodiments, the plug may comprise silicone, polypropylene (PP), polyvinyl alcohol (PVA), ethylene vinyl acetate (EVA), and/or any other biocompatible material. In some embodiments, the suture may be coupled to the plug. In some embodiments, the plug may be inserted into the anchor. In some embodiments, the plug may be inserted into a conduit in the anchor. In some embodiments, the plug may snap into the anchor to secure the plug to the anchor. In some embodiments, a tightness of the suture may be adjusted by sliding the suture into and/or out of the eye through the anchor and/or a cutout in the plug. In some embodiments, the suture may be cut or trimmed, such that the end of the suture is flush with a collar of the anchor. In some embodiments, a knot may be formed in the suture to prevent or inhibit the end of the suture from falling through the anchor and/or the plug and into the eye. In some embodiments, the anchor and/or the plug may remain in the eye after the IOL is inserted (e.g., after the surgical operation). In some embodiments, the collar of the anchor may be the only portion of the system that is exposed between the sclera and the conjunctiva. In some embodiments, only a small portion of the suture may be exposed. Accordingly, in some embodiments, the system may reduce or minimize eroding of the suture.

In some embodiments, the plug may comprise a cutout. In some embodiments, the suture may be coupled to the plug by inserting the suture through the cutout in the plug. In some embodiments, when the plug is positioned in the anchor, the cutout in the plug may secure the suture in place. Accordingly, in some embodiments, the suture may be secured in place without forming a knot in the suture.

In some embodiments, the anchor may comprise a mechanism for securing the plug in the conduit of the anchor. For example, on some embodiments, the anchor may include a projection. In some embodiments, the projection may extend into the conduit of the anchor. In some embodiments, the projection may secure the plug in the conduit. In some embodiments, the anchor may include a recess extending into the anchor. In some embodiments, when the plug is inserted into the conduit of the anchor, the recess may interact with the projection in order to snap the plug. In some embodiments, tension from the suture may apply a force on the plug in order to secure the plug in the conduit of the anchor. Accordingly, the interaction between the recess and the projection may prevent or inhibit the force applied by the suture from pulling the plug out of the anchor and into the eye.

In some embodiments, the cutout in the plug may comprise a tapered recess extending into the plug from an outer wall of the plug. In some embodiments, the cutout may include a conduit extending through the plug. In some embodiments, the cutout may include a wedge shape. In some embodiments, when the plug is inserted into the conduit of the anchor, the conduit may close to grab the suture and secure the suture in the conduit. Accordingly, the suture may be wedged in the cutout secure the suture in the cutout. In some embodiments, a force applied to the suture by the plug may be sufficient to secure the suture in place while also allowing a surgeon to adjust the sutures.

In some embodiments, the plug may include a collar. In some embodiments, the collar may include a cutout extending into the collar. In some embodiments, the cutout may include a wedge shape. In some embodiments, the cutout may be aligned with the cutout in the plug. In some embodiments, the collar may prevent or inhibit the plug from moving too far through the anchor and/or falling into the eye. In some embodiments, the end of the suture may be positioned between the collar of the plug and the collar of the anchor. In some embodiments, the collar may cover the suture. In some embodiments, the collar may provide a smooth surface in order to reduce or minimize irritations of the eye and/or erosion of the suture.

In some embodiments, in order to secure the IOL in place within the eye, two sutures may be coupled to the IOL. In some embodiments, two anchors may be inserted into the eye for each suture coupled to the IOL. Accordingly, in some embodiments, four anchors may be inserted into the eye. In some embodiments, each anchor may receive an end of each suture. In some embodiments, the anchors may be inserted into the eye before the IOL and/or the sutures are inserted into the eye.

In some embodiments, the system may reduce or minimize a time required to perform the surgical operation (e.g., IOL insertion and/or replacement). For example, the system may reduce a 40-minute operation by about 10 minutes to about 15 minutes. In some embodiments, the system may reduce or minimize the technical skill required to perform the surgical operation. In some embodiments, the system may allow the surgeon to secure the IOL more easily in place using sutures. In some embodiments, the system may allow the surgeon to adjust the position of the IOL more easily. In some embodiments, the system may be used by a surgeon for insertion or replacement of any type of IOL. For example, the system may be used for insertion or replacement of a one-piece lens, a 3-piece lens, a haptic lens, and/or any other IOL. In some embodiments, the system may be used for insertion or replacement of any lens that may be secured to the eye with sutures.

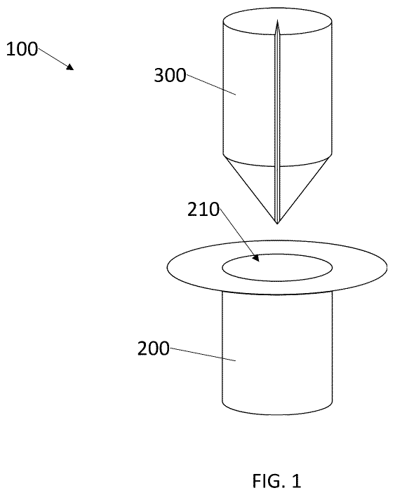

FIG. 1 illustrates a system 100 for securing a suture in an eye according to some embodiments herein. The system 100 may comprise an anchor 200 and a plug 300. The anchor 200 may be inserted into an incision in the eye. The suture may be inserted through a conduit 210 extending through the anchor 200. The plug 300 may be inserted into the conduit 210. The plug 300 may prevent or inhibit the suture from moving out of the conduit 210 of the anchor 200 in order to secure the suture in the eye. In some embodiments, the anchor 200 and/or the plug 300 may comprise silicone, polypropylene (PP), polyvinyl alcohol (PVA), ethylene vinyl acetate (EVA), and/or any other biocompatible material.

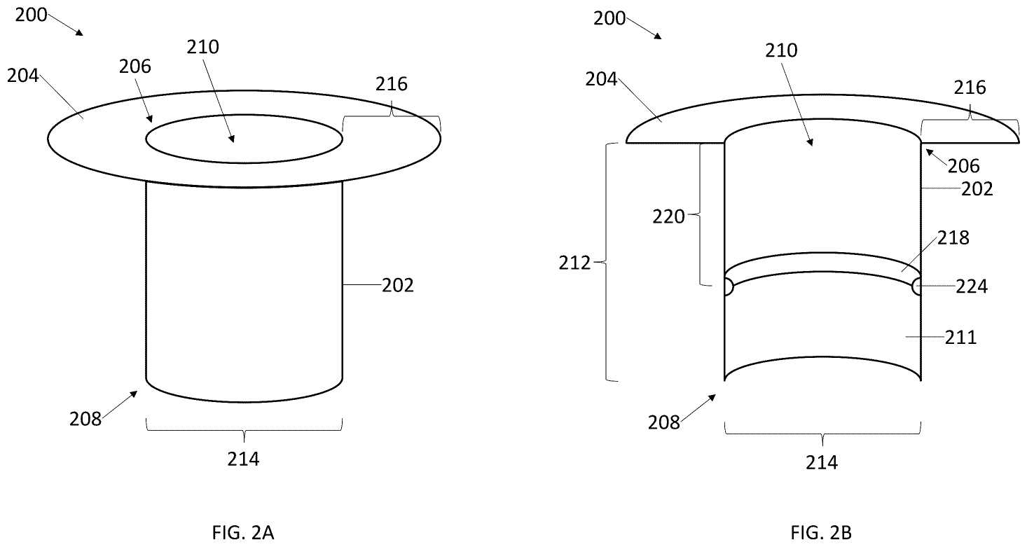

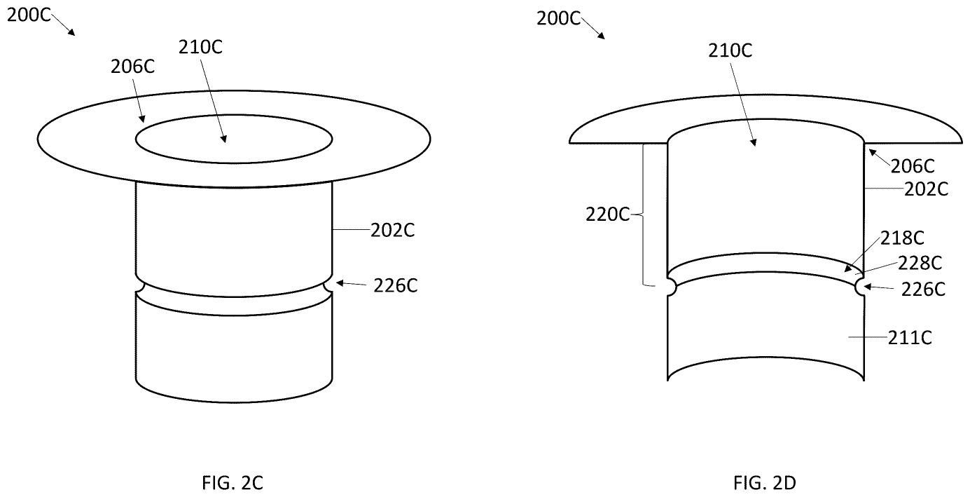

FIGS. 2A and 2B illustrates an example anchor 200 according to some embodiments herein. FIG. 2A illustrates the anchor 200 in isolation. FIG. 2B illustrates a cross-section of the anchor 200. As shown in FIG. 2A, the anchor 200 may comprise a sleeve 202 and a collar 204. The sleeve 202 may extend between a proximal end 206 and a distal end 208. The sleeve 202 and/or the collar 204 may comprise a circular or ovular profile shape. The sleeve 202 may comprise the conduit 210. The conduit 210 may extend through the sleeve 202 from the proximal end 206 to the distal end 208. As shown in FIG. 2B, the sleeve 202 may comprise a height 212. The height 212 of the sleeve 202 may comprise a distance between the proximal end 206 of the sleeve 202 and the distal end 208 of the sleeve 202. The height 212 may comprise a distance of about 0.10 mm, about 0.15 mm, about 0.20 mm, about 0.25 mm, about 0.30 mm, about 0.35 mm, about 0.40 mm, about 0.45 mm, about 0.50 mm, about 0.55 mm, about 0.60 mm, about 0.65 mm, about 0.70 mm, about 0.75 mm, about 0.80 mm, about 0.85 mm, about 0.90 mm, about 0.95 mm, about 1.00 mm, about 1.05 mm, about 1.10 mm, about 1.15 mm, about 1.20 mm, about 1.25 mm, about 1.30 mm, about 1.35 mm, about 1.40 mm, about 1.45 mm, about 1.50 mm, about 1.55 mm, about 1.60 mm, about 1.65 mm, about 1.70 mm, about 1.75 mm, about 1.80 mm, about 1.85 mm, about 1.90 mm, about 1.95 mm, about 2.00 mm, and/or any value between the aforementioned values.

With reference to FIGS. 2A and 2B, the conduit 210 may comprise a width (e.g., radius) 214. The width 214 of the conduit 210 may be sized such that the anchor 200 may be positioned over any trocars and/or cannulas commonly used for intraocular surgical procedures. The width 214 of the conduit 210 may comprise a distance of about 0.20 mm, about 0.25 mm, about 0.30 mm, about 0.35 mm, about 0.40 mm, about 0.45 mm, about 0.50 mm, about 0.55 mm, about 0.60 mm, about 0.65 mm, about 0.70 mm, about 0.75 mm, about 0.80 mm, about 0.85 mm, about 0.90 mm, about 0.95 mm, about 1.00 mm, and/or any value between the aforementioned values.

The collar 204 may be coupled to the sleeve 202 at the proximal end 206 of the sleeve 202. The collar 204 may extend outward from the sleeve 202. The collar 204 may extend radially outward from the proximal end 206 of the sleeve 202. The collar 204 may extend a distance 216 outward from the sleeve 202. The distance 216 may comprise a distance of about 0.05 mm, about 0.10 mm, about 0.15 mm, about 0.20 mm, about 0.25 mm, about 0.30 mm, about 0.35 mm, about 0.40 mm, about 0.45 mm, about 0.50 mm, about 0.55 mm, about 0.60 mm, about 0.65 mm, about 0.70 mm, about 0.75 mm, about 0.80 mm, about 0.85 mm, about 0.90 mm, about 0.95 mm, about 1.00 mm, and/or any value between the aforementioned values.

As shown in FIG. 2B, the anchor 200 may comprise a projection 218. The projection 218 may be positioned in the conduit 210. The projection 218 may be a portion of or coupled to an inner wall (e.g., inner surface) 211 of the sleeve 202. The projection 218 may extend around the inner wall 211 of the sleeve 202. In some embodiments, the projection 218 may extend around a portion of the inner wall 211 of the sleeve 202.

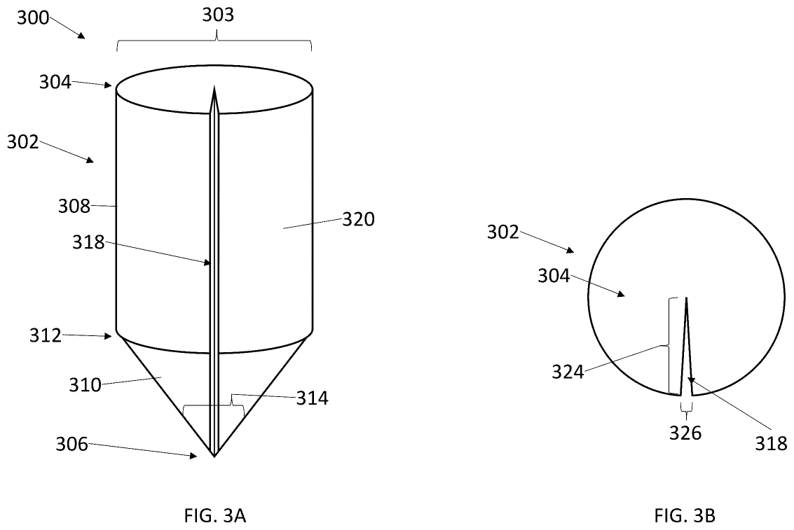

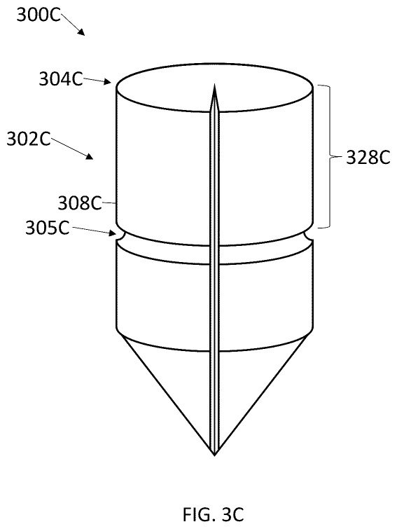

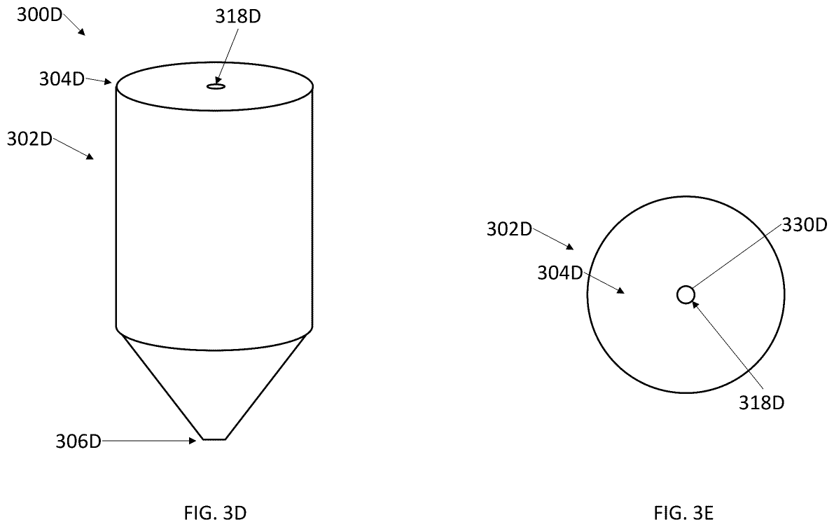

The projection 218 may comprise a profile 224. The profile 224 may comprise a semi-circular shape, a square shape, a triangular shape, and/or any other shape. In some embodiments, the shape of the profile 224 may be selected such that the shape of the profile 224 comprises a same shape as a shape of a recess 305C in a plug 300C (shown in FIG. 3C). The projection 218 may be positioned a distance 220 from the proximal end 206 of the sleeve 202. In some embodiments, the distance 220 may be selected so the projection 218 aligns with the recess 305C in the plug 300C.

The projection 218 may be configured to secure the plug 300 in the conduit 210 in order to couple the plug 300 to the anchor 200. In some embodiments, the projection 218 may engage the recess 305C in the plug 300C, described further below with reference to FIG. 3C, in order to secure the plug 300C in the conduit 210. Accordingly, the plug 300C may snap-fit into the conduit 210. In some embodiments, the plug 300 may friction-fit in the conduit 210. In some embodiments, the projection 218 may apply a force to an outer wall 320 of the plug 300 in order to secure the plug 300 in the conduit 210. Accordingly, friction between the outer wall 320 of the plug 300 and the projection 218 may secure the plug 300 in the conduit 210.

In some embodiments, when the anchor 200 is inserted into an incision in the eye, the collar 204 may be positioned on the conjunctiva and/or sclera. The collar 204 may prevent or inhibit the anchor 200 from falling into the eye through the incision.

FIGS. 2C and 2D illustrate another embodiment of an anchor 200C. The anchor 200C may comprise some or all of the features of the anchor 200. The anchor 200C may comprise a recess 226C in a sleeve 202C of the anchor 200C. The recess 226C may extend into an outer wall (e.g., outer surface) 213C of the sleeve 202C. Accordingly, an inner surface 228C of the recess 226C may form a projection 218C that extends into a conduit 210C from an inner wall 211C of the sleeve 202C. In some embodiments, the recess 226C may facilitate securement of the anchor 200C in an incision in an eye. Tissue of the eye may extend into recess 226C when the anchor 200C is positioned in the incision. Accordingly, the recess 226C may engage with the tissue of the eye to secure the anchor 200C in the incision.

The recess 226C may comprise a semi-circular shape, a square shape, a triangular shape, and/or any other shape. In some embodiments, the shape of the recess 226C may be selected so the shape of the recess 226C comprises a same shape as a shape of the recess 305C in the plug 300C, described further below with reference to FIG. 3C. In some embodiments, the recess 226C may be positioned a distance 220C from the proximal end 206C of the sleeve 202C. In some embodiments, the distance 220C may be selected so the recess 226C and/or the projection 218C align with the recess 305C in the plug 300C when the plug 300C is positioned in the conduit 210C.

FIGS. 3A and 3B illustrate the plug 300. FIG. 3A illustrates the plug 300 in isolation. FIG. 3B illustrates a top view of the plug 300. As shown in FIG. 3A, the plug 300 may comprise a body 302. The body 302 may extend between a proximal end 304 and a distal end 306. The plug 300 may be inserted into the conduit 210 in the sleeve 202 of the anchor 200. The plug 300 may be inserted into the conduit 210 with the distal end 306 inserted first.

The body 302 may comprise a first portion 308 and a second portion 310. The first portion 308 may extend from the proximal end 304 of the body 302 to the second portion 310. The second portion 310 may extend from the first portion 308 to the distal end 306. The first portion 308 may comprise a cylindrical shape, rectangular prism shape, a triangular prism shape, and/or any other shape. The first portion 308 may be shaped so the first portion 308 comprises a same shape as the conduit 210 in the sleeve 202.

The second portion 310 may be tapered from a proximal end 312 of the second portion 310 to the distal end 306 of the body 302. The second portion 310 may form a point at the distal end 306 of the body 302. The second portion 310 may comprise a conical shape, a frustoconical shape, a pyramid shape, and/or any other tapered shape. A width 314 of the second portion 310 at the distal end 306 of the body 302 may be smaller than the width 314 of the second portion 310 at the proximal end 312 of the second portion 310.

The tapered shape of the second portion 310 and/or the point at the distal end 306 of the body 302 may facilitate insertion of the plug 300 into the conduit 210 in the sleeve 202. The width 314 the second portion 310 at the distal end 306 of the body 302 may be smaller than the width 214 of the conduit 210. Therefore, the plug 300 may be inserted into the conduit 210 when only the distal end 306 of the body 302 is aligned with the conduit 210. As the plug 300 is inserted further into the conduit 210, the inner wall 211 of the sleeve 202 may contact the second portion 310 and the tapered shape of the second portion 310 may guide the plug 300 into the conduit 210 and align the first portion 308 of the body 302 with the conduit 210.

The plug 300 may comprise a cutout 318. The cutout 318 may extend into the body 302 from an outer wall 320 of the body 302. As shown in FIG. 3A, the cutout 318 may extend along the body 302 from the proximal end 304 to the distal end 306. As shown in FIG. 3B, the cutout 318 may extend a depth 324 into the body 302. The cutout 318 may be tapered along the depth 324 of the cutout 318. Accordingly, a width 326 of the cutout 318 may decrease along the depth 324 of the cutout 318.

The cutout 318 may be configured to receive a suture. The suture may be coupled to an intraocular lens (IOL). The suture may be positioned in the cutout 318 so the suture extends through the cutout 318 from the distal end 306 of the body 302 to the proximal end 304 of the body 302. When the suture is positioned in the cutout 318 friction between the suture and the body 302 may secure the suture in the cutout 318. The suture may be positioned in the cutout 318 before the plug 300 is inserted into the conduit 210 in the sleeve 202. A width 303 of the first portion 308 of the body 302 may be larger than the width 214 of the conduit 210. Accordingly, when the plug 300 is inserted into the conduit 210, the cutout 318 may close so the first portion 308 of the body 302 may fit in the conduit 210 and the body 302 may clamp onto the suture to secure the suture in the cutout 318. After the plug 300 is inserted into the conduit 210, the suture may be cut and/or tied off in order to prevent or inhibit the suture from moving out of the plug 300 and/or the cutout 318. Accordingly, the plug 300 may secure the suture in the eye so the suture may secure the IOL in place in the eye.

FIG. 3C illustrates another embodiment of a plug 300C. The plug 300C may comprise some or all of the features of the plug 300. The plug 300C may comprise a recess 305C. The recess 305C may extend into a first portion 308C of a body 302C of the plug 300C. The recess 305C may extend laterally (e.g., circumferentially) around the first portion 308C. In some embodiments, the recess 305C may extend around the entire first portion 308C. In some embodiments, the recess 305C may extend around a portion of the first portion 308C. The recess 305C may be configured to receive the projection 218 on the inner wall 211 of the sleeve 202. The projection 218 may engage the recess 305C in order to secure the plug 300C in the conduit 210. Accordingly, the plug 300C may snap-fit into the conduit 210.

The recess 305C may be positioned a distance 328C from a proximal end 304C of the body 302C. In some embodiments, the distance 328C may be selected so the recess 305C aligns with the projection 218 on the inner wall 211 of the sleeve 202. In some embodiments, the distance 328C may be selected so the proximal end 304C of the body 302C aligns with the proximal end 206 of the sleeve 202, when the recess 305C is aligned with the projection 218 on the inner wall 211 of the sleeve 202.

FIGS. 3D and 3E illustrate another embodiment of a plug 300D. FIG. 3D illustrates the plug 300D in isolation. FIG. 3E illustrates a top view of the plug 300D. The plug 300D may comprise some or all of the features of the plugs 300, 300C. The plug 300D may comprise a conduit 318D. The conduit 318D may extend through a body 302D of the plug 300D. The conduit 318D may extend from a proximal end 304D of the body 302D to a distal end 306D of the body 302D. As shown in FIG. 3E, in some embodiments, the conduit 318D may be centered in the body 302D. In some embodiments, the conduit 318D may comprise a profile shape 330D. The profile shape 330D may comprise a circle, an oval, an ellipse, a triangle, a square, a rectangle, and/or any other shape.

The conduit 318D may be configured to perform any of the functions of the cutout 318 of plug 300 described herein. Accordingly, a suture may be inserted through the conduit 318D, such that the suture extends through the conduit 318D from the distal end 306D of the body 302D to the proximal end 304D of the body 302D. After the plug 300D is inserted into the conduit 210 in the sleeve 202 of the anchor 200, the suture may be cut and/or tied off in order to prevent or inhibit the suture from moving out of the plug 300D and/or the conduit 318D.

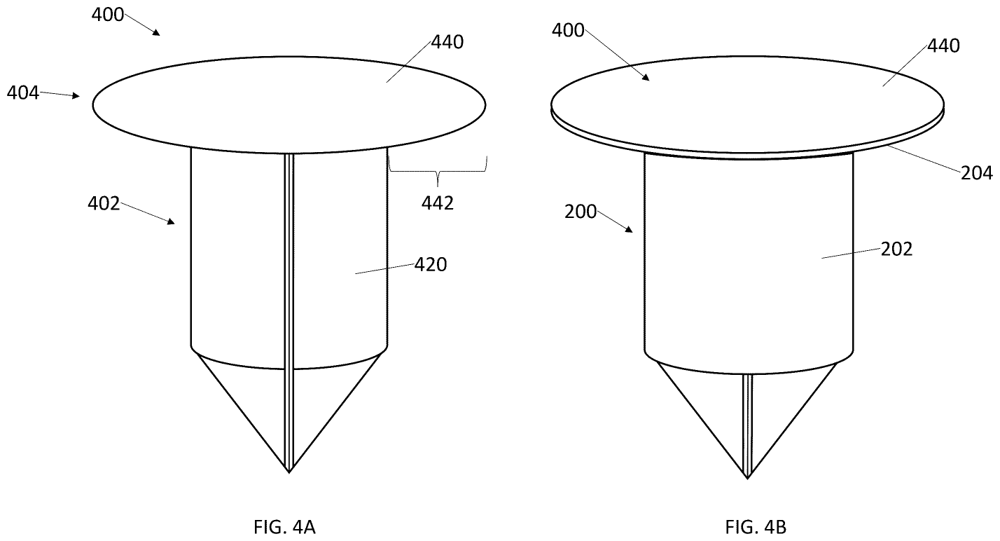

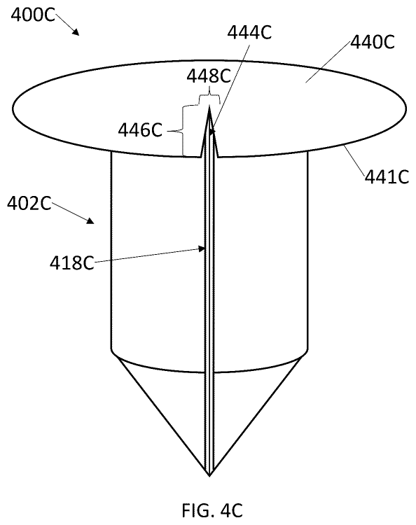

FIGS. 4A and 4B illustrate another embodiment of a plug 400. FIG. 4A illustrates the plug 400 in isolation. FIG. 4B illustrates the plug 400 inserted into the anchor 200. The plug 400 may include some or all of the features of the plugs 300, 300C, 300D. The plug 400 may comprise a collar 440. The collar 440 may comprise a circular or ovular profile shape. The collar 440 may be coupled to a body 402 of the plug 400. The collar 440 may be coupled to the body 402 at a proximal end 404 of the body 402. The collar 440 may extend outward from the body 402. The collar 440 may extend radially outward from the proximal end 404 of the body 402. The collar 440 may extend a distance 442 outward from an outer wall 420 of the body 402. The distance 442 may comprise a distance of about 0.05 mm, about 0.10 mm, about 0.15 mm, about 0.20 mm, about 0.25 mm, about 0.30 mm, about 0.35 mm, about 0.40 mm, about 0.45 mm, about 0.50 mm, about 0.55 mm, about 0.60 mm, about 0.65 mm, about 0.70 mm, about 0.75 mm, about 0.80 mm, about 0.85 mm, about 0.90 mm, about 0.95 mm, about 1.00 mm, and/or any value between the aforementioned values.

As shown in FIG. 4B, when the plug 400 is positioned in the anchor 200, the collar 440 may be aligned with the collar 204 of the anchor 200. The collar 440 of the plug 400 may contact the collar 204 of the anchor 200 in order to prevent or inhibit the plug 400 from moving through the sleeve 202 of the anchor 200 (e.g., in a direction towards the bottom of FIG. 4B). The end of the suture may be positioned between the collar 440 of the plug 400 and the collar 204 of the anchor 200. The collar 440 of the plug 400 may cover the end of the suture. The collar 440 of the plug 400 may provide a smooth surface when the plug 400 is positioned in the anchor 200. Accordingly, the collar 440 of the plug 400 may reduce or minimize irritation of the eye and/or erosion of the suture.

FIG. 4C illustrated another embodiment of a plug 400C. The plug 400C may include some or all of the features of the plugs 300, 300C, 300D, 400. A collar 440C of the plug 400C may include a cutout 444C. The cutout 444C may extend into the collar 440C from an outer edge 441C of the collar 440C. The cutout 444C may extend a depth 446C into the collar 440C. The cutout 444C may be tapered along the depth 446C of the cutout 444C. Accordingly, a width 448C of the cutout 444C may decrease along the depth 446C of the cutout 444C. The cutout 444C may be aligned with a cutout 418C in the body 402C of the plug 400C.

The cutout 444C may provide an opening in the collar 440C so the collar 440C does not cover the cutout 418C in the body 402C of the plug 400C and/or prevent or inhibit the suture from being inserted through the cutout 418C in the body 402C of the plug 400C. In some embodiments, after the suture is cut and/or tied off an end of the suture may be positioned in the cutout 444C. In some embodiments, the end of the suture may be positioned between the collar 440C and the collar 204 of the anchor 200 so the end of the suture does not extend above the collar 440C.

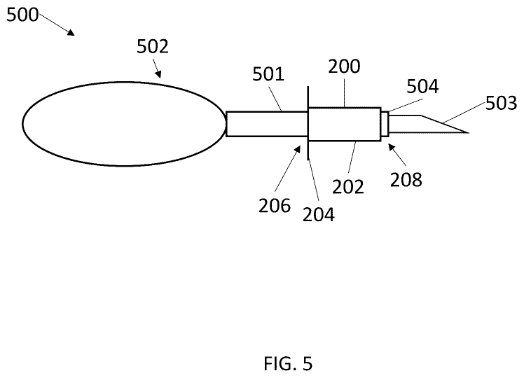

FIG. 5 illustrates a system 500 for inserting the anchor 200 in an eye. In some embodiments, the system 500 may include a trocar 502. In some embodiments, the trocar 502 may comprise a 23-gauge trocar, a 25-gauge trocar, a 27-gauge trocar, and/or any other trocar used for intraocular surgical operations. In some embodiments, the system 500 may include a cannula 504. The cannula 504 may be positioned over an awl portion 501 of the trocar 502. The anchor 200 may be positioned over the awl portion 501 of the trocar 502 so the anchor 200 is positioned over the cannula 504. Accordingly, the cannula 504 may be positioned in the conduit 210 (shown in FIGS. 2A and 2B) of the anchor 200.

The awl portion 501 of the trocar 502 may be inserted into the eye. The awl portion 501 may be inserted into the eye while the anchor 200 and/or the cannula 504 are positioned over the awl portion 501. The anchor 200 may be positioned on the awl portion 501 so the distal end 208 of the sleeve 202 of the anchor 200 is positioned closer to a tip 503 of the awl portion 501 than the proximal end 206. Accordingly, the distal end 208 of the sleeve 202 may be inserted into the eye first. Therefore, the proximal end 206 of the sleeve 202 and/or the collar 204 may be positioned outside of the eye and/or on the conjunctiva or sclera of the eye.

The tip 503 of the awl portion 501 may form an incision in the eye when the awl portion 501 is inserted into the eye. The awl portion 501 may be inserted into the eye so the anchor 200 and/or the cannula 504 are positioned in the insertion. When the awl portion 501 of the trocar 502 is removed from the incision, the anchor 200 and/or the cannula 504 may remain in the incision.

One or more surgical tools may be inserted through the cannula 504 in order to perform one or more steps of an intraocular surgical operation. After the one or more steps of the intraocular surgical operation are completed, the cannula 504 may be removed from the conduit 210 of the anchor 200. The anchor 200 may remain in the incision after the cannula 504 is removed from the conduit of the anchor 200.

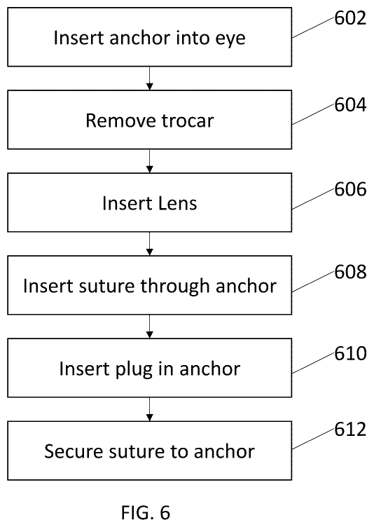

FIG. 6 illustrates a method 600 of performing an intraocular surgical operation with the system 100. In some embodiments, the intraocular surgical operation may comprise inserting and/or replacing an IOL in the eye.

In some embodiments, at step 602, the anchor 200 may be inserted into the eye. The anchor 200 may be inserted into the eye so the anchor 200 is positioned in the sclera of the eye. As described above with reference to FIG. 5, the anchor 200 may positioned over the awl portion 501 of the trocar 502 and/or a cannula 504. The awl portion 501 of the trocar 502 may be inserted into the eye so the anchor 200 and/or the cannula 504 are positioned in an insertion in the eye. In some embodiments, the awl portion 501 may be inserted into the eye without making a separate incision in the conjunctiva of the eye before inserting the awl portion 501. Accordingly, the anchor 200 and/or the cannula 504 may be inserted into the eye without making the separate incision in the conjunctiva before the anchor 200 and/or the cannula 504 are inserted into the eye.

In some embodiments, at step 604, the trocar 502 may be removed from the eye. When the trocar 502 is removed from the eye, the anchor 200 and/or the cannula 504 may remain in the incision in the eye.

In some embodiments, steps 602 and 604 may be repeated one or more times in order to insert a desired number of anchors 200 and/or cannulas 504 into the eye. In some embodiments, the desired number of anchors 200 and/or cannulas 504 may comprise a number of sutures coupled to the IOL. In some embodiments, the desired number of anchors 200 and/or cannulas 504 may comprise twice the number of sutures coupled to the IOL. In some embodiments, the desired number of anchors 200 and/or cannulas 504 may comprise two (2), three (3), four (4), five (5), six (6), seven (7), eight (8), nine (9), and/or ten (10). Therefore, steps 602 and 604 may be repeated one (1) time, two (2) times, three (3) times, four (4) times, five (5) times, six (6) times, seven (7) times, eight (8) times, and/or nine (9) times.

In some embodiments, at step 606, an IOL may be inserted into the eye. In some embodiments, one or more surgical instruments may be inserted into the eye through the anchor 200 and/or the cannula 504. In some embodiments, before the IOL is inserted into the eye, one or more sutures may be coupled to the IOL. The sutures may be configured to secure the IOL in place in the eye. In some embodiments, two anchors 200 and/or cannulas 504 may be inserted into the eye for each suture. For example, if two sutures are used to secure the IOL in place in the eye, four anchors 200 and/or cannulas 504 may be inserted into the eye. In some embodiments, the number of sutures coupled to the IOL may be equal to the number of anchors 200 and/or cannulas 504 inserted into the eye.

In some embodiments, at step 608, the suture may be inserted through the anchor 200. The suture may be inserted through the conduit 210 in the sleeve 202 of the anchor 200. As described above, the suture may be coupled to the IOL. Therefore, the suture may be positioned in the eye. The suture may be inserted through the anchor 200 from the distal end 208 of the sleeve 202 of the anchor 200 to the proximal end 206 of the sleeve 202 of the anchor 200. Accordingly, the suture may extend out of the eye through the anchor 200. In some embodiments, the cannula 504 may be removed from the eye and/or the anchor 200 before the suture is inserted through the anchor 200 at step 608. In some embodiments, the cannula 504 may be removed from the eye and/or the anchor 200 after the suture is inserted through the anchor 200 at step 608. In some embodiments, the cannula 504 may be removed from the eye and/or the anchor 200 while the suture is inserted through the anchor 200 at step 608.

In some embodiments, at step 610, the plug 300 may be inserted into the anchor 200. The plug 300 may be inserted into the conduit 210 of the anchor 200. In some embodiments, the suture may be inserted into the cutout 318 of the plug 300 before the plug 300 is inserted into the anchor 200 at step 610. In some embodiments, the suture may be inserted into the cutout 318 while the plug 300 is being inserted into the anchor 200.

In some embodiments, at step 612, the suture may be secured to the anchor 200 and/or the plug 300. In some embodiments, the suture may be tied-off (e.g., knotted) to secure the suture to the anchor 200 and/or the plug 300. The suture may be tied-off to form a knot in the suture. The knot may prevent or inhibit the suture from moving out of the cutout 318 in the plug 300 and into the eye. Therefore, the knot may secure the suture in place in the anchor 200 and/or the plug 300. Accordingly, the suture may secure the IOL in place in the eye.

In some embodiments, steps 608-612 may be repeated for each anchor 200 inserted into the eye in steps 602 and 604. In some embodiments, steps 608-612 may be performed for each anchor 200 before steps 608-612 are performed for subsequent anchors 200. In some embodiments, each step of steps 608-612 may be performed for each anchor 200 before the subsequent step of steps 608-612 are performed for each anchor 200. For example, step 608 may be performed for each anchor 200 before step 610 is performed for each anchor, and step 610 may be performed for each anchor before step 612 is performed for each anchor.

It is to be appreciated that although the specification describes the system 100, the anchors 200, 200C, the plugs 300, 300C, 300D, 400, 400C, and the method 600 with reference to IOLs, the system 100, the anchors 200, 200C, the plugs 300, 300C, 300D, 400, 400C and/or the method 600 may be used to secure any device with a suture, or suture-like appendage in the eye without departing from the scope of the disclosure. Such devices with a suture or suture-like appendage may include, but are not limited to, intravitreal inserts for medication, such as ganciclovir implants, long-release steroid implants, gene therapy vector implants, scaffolds for genetically modified cells that release therapeutic agents (e.g., Neurotech's NT501), intraocular lenses, such as 3-piece lenses with haptics comprising Prolene™ and/or other suitable materials, intraocular lenses suitable for use with the Yamane technique or the like.

### Additional Embodiments

Conditional language used herein, such as, among others, “can,” “could,” “might,” “may,” “e.g.,” and the like, unless specifically stated otherwise, or otherwise understood within the context as used, is generally intended to convey that certain configurations include, while other configurations do not include, certain features, elements and/or states. Thus, such conditional language is not generally intended to imply that features, elements and/or states are in any way required for one or more configurations or that one or more configurations necessarily include these features, elements and/or states.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain configurations require the presence of at least one of X, at least one of Y, and at least one of Z.

While the above detailed description may have shown, described, and pointed out novel features as applied to various configurations, it may be understood that various omissions, substitutions, and/or changes in the form and details of any particular configuration may be made without departing from the spirit of the disclosure. As may be recognized, certain configurations may be embodied within a form that does not provide all of the features and benefits set forth herein, as some features may be used or practiced separately from others.

Additionally, features described in connection with one configuration can be incorporated into another of the disclosed configurations, even if not expressly discussed herein, and configurations having the combination of features still fall within the scope of the disclosure. For example, features described above in connection with one configuration can be used with a different configuration described herein and the combination still fall within the scope of the disclosure.

It should be understood that various features and aspects of the disclosed configurations can be combined with, or substituted for, one another in order to form varying modes of the configurations of the disclosure. Thus, it is intended that the scope of the disclosure herein should not be limited by the particular configurations described above. Accordingly, unless otherwise stated, or unless clearly incompatible, each configuration of this disclosure may comprise, additional to its essential features described herein, one or more features as described herein from each other configuration disclosed herein.

Features, materials, characteristics, or groups described in conjunction with a particular aspect, configuration, or example are to be understood to be applicable to any other aspect, configuration or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing configurations. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some configurations, the actual steps taken in the processes illustrated and/or disclosed may differ from those shown in the figures. Depending on the configuration, certain of the steps described above may be removed, others may be added.

Furthermore, the features and attributes of the specific configurations disclosed above may be combined in diverse ways to form additional configurations, all of which fall within the scope of the present disclosure. Also, the separation of various system components in the implementations described above should not be understood as requiring such separation in all implementations, and it should be understood that the described components and systems can generally be integrated together in a solitary product or packaged into multiple products.

For purposes of this disclosure, certain aspects, advantages, and novel features are described herein. Not necessarily all such advantages may be achieved in accordance with any particular configuration. Thus, for example, those skilled in the art will recognize that the disclosure may be embodied or conducted in a manner that achieves one advantage or a group of advantages as taught herein without necessarily achieving other advantages as may be taught or suggested herein.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately,” “about,” “generally,” and “substantially” may refer to an amount that is within less than 10% of, within less than 5% of, within less than 1% of, within less than 0.1% of, and within less than 0.01% of the stated amount. As another example, in certain configurations, the terms “generally parallel” and “substantially parallel” refer to a value, amount, or characteristic that departs from exactly parallel by less than or equal to 15 degrees, 10 degrees, 5 degrees, 3 degrees, 1 degree, 0.1 degree, or otherwise.

The scope of the present disclosure is not intended to be limited by the specific disclosures of preferred configurations in this section or elsewhere in this specification and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

Unless the context clearly requires otherwise, throughout the description and the claims, the words “comprise,” “comprising,” and the like, are to be construed in an inclusive sense as opposed to an exclusive or exhaustive sense, which is to say, in the sense of “including, but not limited to.”

Reference to any prior art in this description is not, and should not be taken as, an acknowledgement or any form of suggestion that that prior art forms part of the common general knowledge in the field of endeavor in any country in the world.

The invention may also be said broadly to consist in the parts, elements and features referred to or indicated in the description of the application, individually or collectively, in any or all combinations of two or more of said parts, elements or features.

Where, in the foregoing description, reference has been made to integers or components having known equivalents thereof, those integers are herein incorporated as if individually set forth. In addition, where the term “substantially” or any of its variants have been used as a word of approximation adjacent to a numerical value or range, it is intended to provide sufficient flexibility in the adjacent numerical value or range that encompasses standard manufacturing tolerances and/or rounding to the next significant figure, whichever is greater.

It should be noted that various changes and modifications to the presently preferred configurations described herein will be apparent to those skilled in the art. Such changes and modifications may be made without departing from the spirit and scope of the invention and without diminishing its attendant advantages. For instance, various components may be repositioned as desired. It is therefore intended that such changes and modifications be included within the scope of the invention. Moreover, not all of the features, aspects and advantages are necessarily required to practice the present invention. Accordingly, the scope of the present invention is intended to be defined only by the claims.

What is claimed is:

1. A method for securing a suture to an eye, the method comprising the steps of:
inserting a trocar through the eye to form an incision in the eye, wherein a cannula is positioned over the trocar and an anchor is positioned over the cannula such that the cannula is positioned in a conduit extending through the anchor;
removing the trocar from the eye such that the anchor and the cannula remain in the incision;
removing the cannula from the conduit in the anchor;
inserting an end of the suture through the anchor; and
securing the suture to the anchor.

2. The method of claim 1, wherein securing the suture to the anchor further comprises:
inserting the suture through a cutout in a plug of the anchor; and
inserting the plug into the conduit in the anchor.

3. The method of claim 2, wherein securing the suture to the anchor further comprises knotting the end of the suture and/or trimming the suture.

4. The method of claim 2, wherein the cutout is tapered along a depth of the cutout.

5. The method of claim 1, further comprising:
inserting a second trocar through the eye to form a second incision in the eye, wherein a second cannula is positioned over the second trocar and a second anchor is positioned over the second cannula such that the second cannula is positioned in a conduit extending through the second anchor;
removing the second trocar from the eye such that the second anchor and the second cannula remain in the second incision;
removing the second cannula from the conduit in the second anchor;
inserting a second end of the suture through the second anchor; and
securing the suture to the anchor.

6. An anchor for securing a suture to an eye, the anchor comprising:
a sleeve comprising a conduit extending through the sleeve;
a collar extending from a proximal end of the sleeve,
wherein the sleeve is configured to receive a plug in the conduit extending through the sleeve, the plug configured to couple the suture to the anchor when the sleeve is positioned in an incision in the eye.

7. The anchor of claim 6, wherein the sleeve and/or the collar comprise silicone.

8. The anchor of claim 6, further comprising a projection extending into the conduit from an inner wall of the sleeve.

9. The anchor of claim 6, wherein the sleeve is configured to be inserted into the incision in the eye with a trocar.

10. A system for securing a suture to an eye, the system comprising:
an anchor configured to be inserted into an incision in the eye, the anchor comprising a conduit;
a plug configured to be inserted into the conduit, wherein the plug is configured to secure the suture in place when the plug is inserted into the conduit.

11. The system of claim 10, wherein the anchor and the plug comprise silicone.

12. The system of claim 10, wherein the anchor comprises a sleeve and a collar extending from the sleeve, and wherein the conduit extends through the sleeve.

13. The system of claim 12, wherein the anchor comprises a projection extending into the conduit, and wherein the projection is configured to secure the plug in the anchor.

14. The system of claim 13, wherein the plug comprises a recess configured to receive the projection when the anchor is positioned in the conduit.

15. The system of claim 10, wherein a distal end of the plug is inserted into the conduit first, and wherein the plug is tapered at the distal end.

16. The system of claim 10, wherein the plug comprises a cutout configured to receive the suture.

17. The system of claim 16, wherein the cutout extends through the plug from a proximal end of the plug to a distal end of the plug.

18. The system of claim 10, wherein the plug comprises a collar.

19. The system of claim 18, wherein the collar extends radially outward from a proximal end of the plug.

20. The system of claim 18, wherein the collar comprises a cutout.
